(19) United States
(12) Patent Application Publication
(10) Pub. No.: US 2009/0209985 A1
(43) Pub. Date: Aug. 20, 2009

(54) Sensor and pacemaker imbedded with a gastric banding

(76) Inventor: Teddy Khalili, Los Angles, CA (US)

Correspondence Address: SOFER & HAROUN LLP., 317 MADISON AVENUE, SUITE 910, NEW YORK, NY, 10017, US

(21) Appl. No.: 12/284,472

(22) Filed: Sep. 22, 2008

Related U.S. Application Data
(60) Provisional application No. 60/994,667, filed on Sep. 20, 2007.

(51) Int. Cl. A61N 1/36; A61B 17/12

(52) U.S. Cl. 606/157; 607/40

(57) ABSTRACT

A device for controlling food intake including a band, is configured to be placed around the stomach of a patient, dividing the stomach into lower and upper pouches. At least one sensor, located on the band detects pressure against the band caused by food ingestion collected in the upper pouch. A controller is coupled to the senor for receiving signals from the sensor for comparison against a predefined threshold. At least one electrode is coupled to the controller and the stomach, where the controller, upon the detection of pressure in the sensor above the pre-defined threshold, generates an electronic interference signal to the stomach of the patient.

### RELATED APPLICATIONS

This application claims the benefit of priority from U.S. Provisional Patent Application No. 60/994,667, filed on Sep. 20, 2007, the entirety of which is incorporated herein by reference.

### FIELD OF THE INVENTION

This application relates to surgical devices. More particularly, the present invention relates to an improved gastric banding device.

### BACKGROUND

Peristalsis, the transit of luminal contents is accomplished by coordinated contractions in the stomach. The stomach also requires coordinated gastric contractions for normal emptying. Gastric contractions are regulated by the myoelectrical activity of the stomach. The rate of gastric emptying affects food intake. Obese individuals tend to have a more rapid gastric emptying rate than the non-obese.

Implantable gastric stimulation is a mechanism employed to facilitate weight loss in obese individuals. This treatment is unique in that it involves the least invasive surgery and does not alter the gastrointestinal tract anatomy. Electrical gastric pacing can retard contractions in the stomach and slow down gastric emptying. As a treatment for obesity, the delay in gastric emptying leads to early satiety and decreased food intake.

FIG. 1 illustrates an implantable gastric stimulation system in accordance with the prior art. A pacer or pulse generator 12 is embedded in a patient's body just below the skin. Pacer 12 includes electrodes 14 that are attached to stomach 16 in a particular location 18 of the stomach. A second set of leads 20 are attached to the stomach and act as electrical sensors to estimate the signal activity in stomach 16, in particular whether peristaltic contractions are occurring.

In operation, the electrical stimulation of the gastrointestinal tract is governed by implantable pulse generator 12 featuring the two sets of leads 14 and 20 which are coupled to stomach 16. Leads 14 provide stimulation pulses from pulse generator 12 to stomach 16. Leads 20 provide sensing of the gastroelectrical activity of stomach 16 and convey the sensed signal to pulse generator 12. Pulse generator 12 then generates output signals when the sensed input signal is above a threshold level.

The electronic leads 14, 20 of pulse generator 12 are sutured into the outside of stomach 16 as electrical impulses are sent to the smooth muscle of stomach 16 causing a halt of the natural movement of food from the stomach to the small bowel, thereby retarding gastric emptying. The waves generated by this artificial process conflict with the normal physiological electrical waves that propagate from the stomach and regulate gastric contractions. This causes the patient to feel full and satisfied, even with lower food intake than they are normally accustomed to. This method is described in greater detail in U.S. Pat. No. 6,115,635 to Bourgeois, incorporated herein by reference.

Another obesity treatment known in the prior art, shown in FIG. 2 is an arrangement for adjustable gastric banding. As illustrated, this arrangement includes a band 22 placed around stomach 16 in order to induce a feeling of satiety. This band divides the stomach into a small upper pouch 26 and a larger lower pouch 28 below the band. The small gastric pouch 26 limits the amount of food that the patient can eat at one time before it passes through band 22 to lower pouch 28, and results in a feeling of fullness after eating a small amount.

In such an arrangement, when the patient does eat, there is a sense of rapidly achieved fullness. If the patient continues to eat, they develop nausea, vomiting, or pain which generally results in the termination of eating. Patients must adapt to their new stomach size and restrict their food intake to prevent these side effects. In some cases, even if patients have not over-eaten, they do not feel hungry.

Band 22 is removable, adjustable and does not alter the anatomy of the patient. The precise placement of band 22 at the top of the stomach is critical. Recently a new surgical technique referred to as Laparoscopic gastric banding is employed which utilizes a laparoscope, an instrument which is inserted into the abdominal cavity. Five to six tiny incisions are made in order to implant the device. By avoiding the large incision of open surgery, patients generally experience less pain and scarring. Because no permanent changes are made to the body, the procedure can be reversed and the stomach can return to its original physiologic function once the band is removed.

There are, however, some disadvantages that come with adjustable gastric banding. Gastric banding patients can and do cheat the system. Patients can still eat excessively and push the food particles past the small outlet created by the band. Another common means of cheating the system is drinking high calorie liquids such as milk shakes between or during meals. As a single mechanism therapy that is merely restrictive, many patients report significant continued hunger cravings and resort to the ingestion of empty calories in the form of milkshakes, etc.

### OBJECTS AND SUMMARY

The present invention looks to overcome the drawbacks associated with the prior art and to provide an improved method for treating obese patients. Such an arrangement employs a gastric band with a coupled electronic pacemaker.

To this end, the present invention provides for a device for controlling food intake including a band, configured to be placed around the stomach of a patient, dividing the stomach into lower and upper pouches. At least one sensor, located on the band detects pressure against the band caused by food ingestion collected in the upper pouch. A controller is coupled to the senor for receiving signals from the sensor for comparison against a predefined threshold. At least one electrode is coupled to the controller and the stomach, where the controller, upon the detection of pressure in the sensor above the pre-defined threshold, generates an electronic interference signal to the stomach of the patient.

### BRIEF DESCRIPTION OF THE DRAWINGS

The present invention can be best understood through the following description and accompanying drawings, wherein:

FIG. 1 illustrates a prior art gastric pacing device;

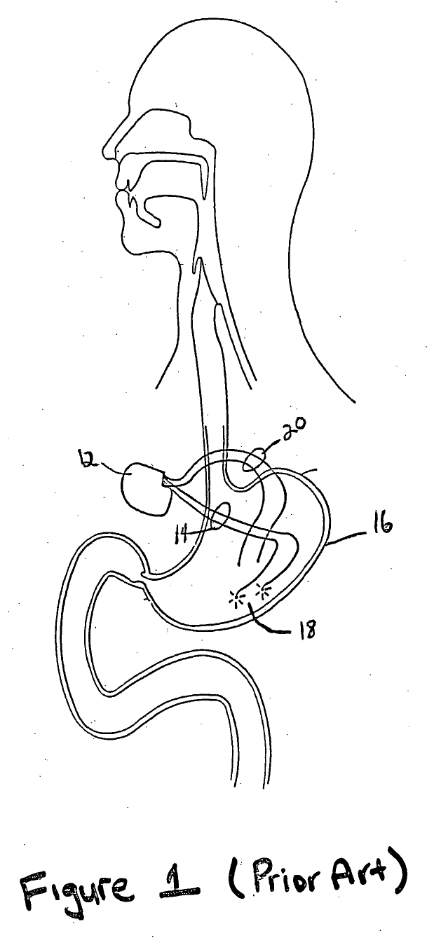

FIG. 2 illustrates a prior art gastric banding device;

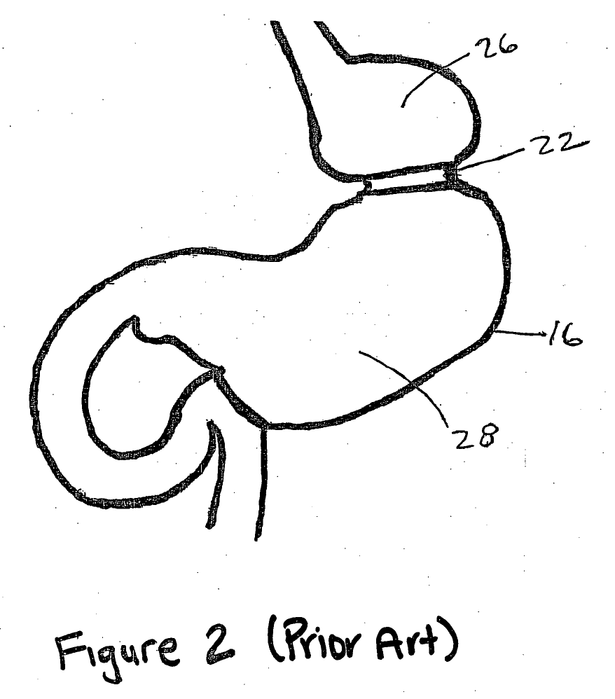

FIG. 3 is an illustration of an improved banding device, in accordance with one embodiment of the present invention;

FIG. 4 is an illustration of an improved banding device, in accordance with another embodiment of the present invention;

FIG. 5 is an illustration of an improved banding device, in accordance with another embodiment of the present invention; and

FIG. 6 is an illustration of an improved banding device, in accordance with another embodiment of the present invention.

### DETAILED DESCRIPTION

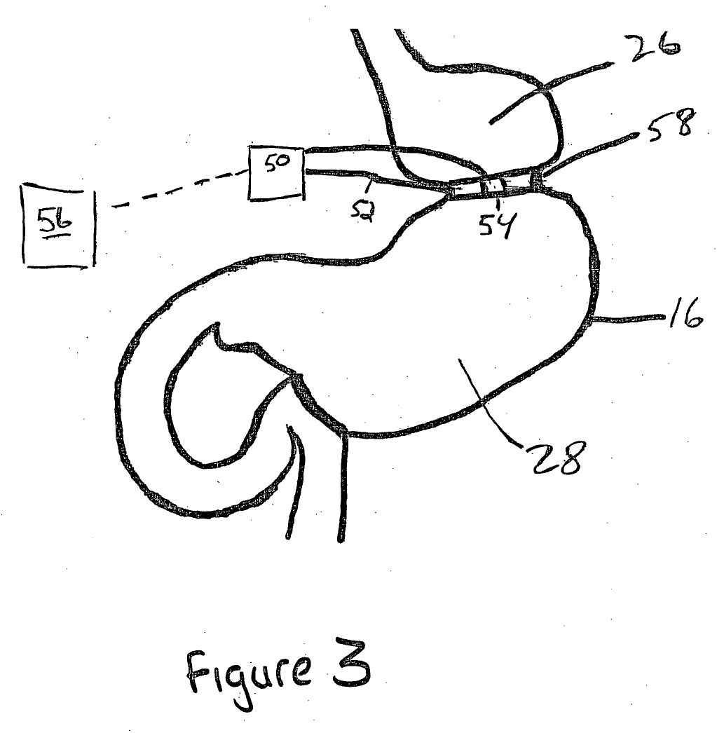

In one embodiment of the present invention, as illustrated in FIG. 3, the present invention includes a controller or electronic pacemaker unit 50; a plurality of electrodes 52 which carry electrical signals; an electrode sensor 54; a programming device 56 and a gastric band 58. Controller unit 50 is placed beneath the skin and is connected to two sets of electrodes 52. The sensing mechanism 54 of the present invention is different from the prior art in that the prior art senses gastro-electrical activity, while the electrode sensor 54 of the present invention is a pressure sensor detecting the pressure changes in the stomach due to the exertion of the gastric band. The change in pressure exerted on band 58 due to food intake is sensed by electrode sensor 54 and in accordance with a prescribed pressure threshold, which in turn sends a signal to controller unit 50 which signals electrodes 52 to emit electrical waves into the stomach.

The administering physician is capable of regulating the system via programming device 56 which remains outside the body and communicates electronically, preferably wirelessly, with controller unit 50. If a patient does not experience a feeling of satiety, then the doctor uses the programming device to adjust the thresholds for sensor 54 and triggering electrodes 52. The frequency and amplitude of controller unit 50 may be adjusted by programming device 56 so that it can fire electrical signals at different thresholds for different patients. Patients may be fed a small meal and controller unit 50 is adjusted to induce satiety as a result of a small meal.

In accordance with one embodiment of the present invention, as illustrated in FIG. 3, the arrangement includes electrode sensor 54 and emitting electrode 52 are embedded within gastric band 58 itself for sending the interference signals to stomach 16. Various algorithms for using the system can be programmed by the physician by using programming device 56. For example, controller unit 50 fires electrical waves at a baseline rate frequency through electrodes 52 and stops firing when a threshold pressure is detected along gastric band 58 by sensor 56. In another manner of operation for this arrangement, electrodes 52 fire only when a threshold pressure is reached along band 58 and fires for a predetermined amount of time or until the pressure on band 58 returns to normal.

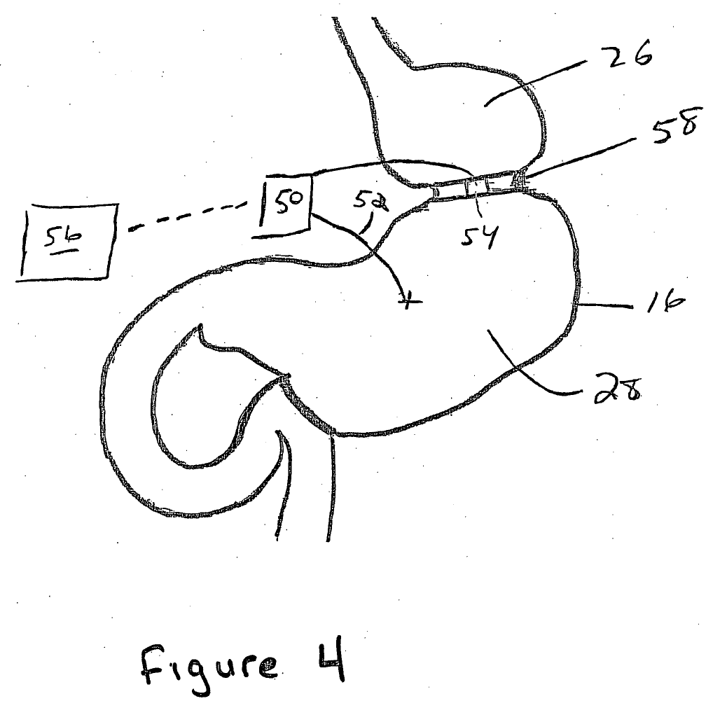

In a second embodiment of the present invention, as shown in FIG. 4, emitting electrodes 52, may be imbedded separately from band 58, along the outer wall of stomach 16 in lower pouch 28 with sensor electrode 54 imbedded within band 58 itself.

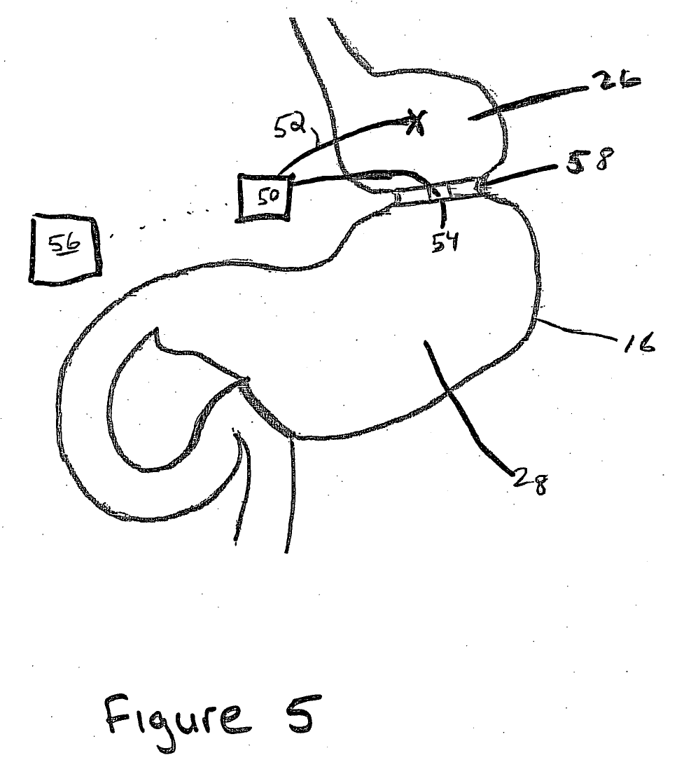

In a third embodiment of the present invention, as shown in FIG. 5, emitting electrodes 52 may be imbedded within the upper pouch 26 of stomach 16 located above band 58 with sensor 54 remaining within band 58 in the distal stomach as depicted in FIG. 5.

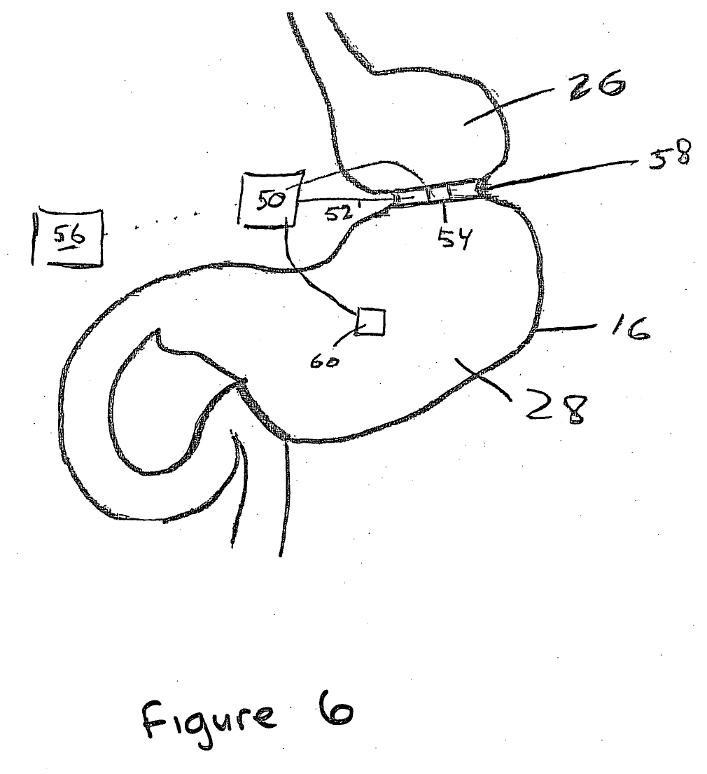

In yet another embodiment of the present invention, as illustrated in FIG. 6, an additional sensor 60 may be employed. In this arrangement, controller unit 50 receives signals from both pressure sensor 54 in band 58 as well as a gastro-electrical sensor 60 located in lower pouch 28 of stomach 16. Based on this combined information, controller unit 50 may emit signals to electrode 52, in either band 58 (as shown) or in upper pouch 26 (not shown) to emit the electrical waves to the stomach.

Thus, the arrangement of the present invention does not rely primarily on a band alone or on electrical sensor/signal but instead combines the system by applying a pressure sensor to band 58 which is used to detect eating necessary to trigger the disruption electrical signals sent over electrodes 52.

While only certain features of the invention have been illustrated and described herein, many modifications, substitutions, changes or equivalents will now occur to those skilled in the art. It is therefore, to be understood that this application is intended to cover all such modifications and changes that fall within the true spirit of the invention.

What is claimed is:

1. A device for controlling food intake, said device comprising:
a band, configured to be placed around the stomach of a patient, dividing the stomach into lower and upper pouches;
at least on sensor, located on said band, configured to detect pressure against said band caused by food ingestion collected in said upper pouch;
a controller coupled to said senor configured to receive signals from said sensor for comparison against a predefined threshold; and
at least one electrode coupled to said controller and the stomach, wherein said controller, upon the detection of pressure in said sensor above said pre-defined threshold, generates an electronic interference signal to the stomach of said patient.

2. The device as claimed in claim 1, further comprising a second sensor coupled to the lower pouch of said stomach coupled to said controller.

3. The device as claimed in claim 2, wherein said second sensor is a gastro-electrical sensor.

4. The device as claimed in claim 1, wherein said at least one electrode is coupled directly to said band.

5. The device as claimed in claim 1, wherein said at least one electrode is coupled to the upper pouch of the stomach.

6. The device as claimed in claim 1, wherein said at least one electrode is coupled to the lower pouch of the stomach.

7. The device as claimed in claim 1, further comprising a programming device configured to allow a user to adjust the pre-determined threshold for said pressure sensor.

8. The device as claimed in claim 7, wherein said programming device communicates with said controller wirelessly.
